(19) United States
(12) Patent Application Publication
(10) Pub. No.: US 2007/0048230 A1
(43) Pub. Date: Mar. 1, 2007

(54) Pheromone compositions and methods

(76) Inventors: Walter Parsadayan, Laguna Niguel, CA (US); Amber Rose, Laguna Beach, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/211,227

(22) Filed: Aug. 24, 2005

(51) Int. Cl. A61K 9/14; A61K 31/57; A61L 9/04

(52) U.S. Cl. 424/46; 514/177; 128/200.23

(57) ABSTRACT

Compositions and methods according to the inventive subject matter make use of human pheromones, and particularly androst-16-en steroids to achieve a desired non-behavioral effect in a person exposed to such compositions. Most preferably, contemplated compositions increase exercise strength and/or stamina, and reduce airway inflammation (e.g., sinusitis). Typically, the composition is a dry composition in a container that allows evaporation of the pheromone at a rate effective to achieve the non-behavioral effect.

### FIELD OF THE INVENTION

The field of the invention is human pheromones, especially as they relate to generation of a non-behavioral response in a human.

### BACKGROUND OF THE INVENTION

Human pheromones, and especially human steroid pheromones are well known in the art and have been associated with generation of various behavioral responses. For example, certain of the human pheromones were reported to alleviate anxiety, promote beneficial moods, and to alter hypothalamic functions (e.g., modify satiety level, energy balance, and/or reproductive functions) as described in U.S. Pat. App. No. 2003/0049726 and U.S. Pat. No. 6,432,938). Still further published behavioral effects include an increase in transgender attraction as described by Friebely et al. (J. Sex. Res. 2004 November; 41(4):372-80) In yet other examples, human pheromones were also described as alleviating symptoms of PMS and anxiety as taught in U.S. Pat. No. 6,057,439. Only few non-behavioral responses due to human pheromone exposure are known in the art. Among other things, human pheromones were proposed to treat paroxistic tachycardia as described in U.S. Pat. No. 6,117,860, and to achieve a change in luteinizing hormone levels in human blood levels of as disclosed in U.S. Pat. No. 6,066,627.

Despite the relative well known receptor for human pheromones (vomeronasal receptor, see e.g., Physiol. Behav. 2004 Nov. 15; 83(2):177-87, or Prog. Neurobiol. 2003 June; 70(3):245-318), the mode of action of human pheromones is not entirely understood. Among various alternative mechanisms, receptor binding may was reported to affect neurobiochemical aspects (see e.g., Microsc. Res. Tech. 2002 Aug. 1; 58(3):251-60) as well as hormone-receptor mediated effects that alter transcription of hormone responsive genes or gene clusters.

Therefore, while there are numerous compositions and methods for human pheromones are known in the art, the full potential of human pheromones is likely not realized at present. Thus, there is still a need for new compositions and methods for human pheromones, and especially for human steroid pheromones.

### SUMMARY OF THE INVENTION

The present invention is directed to compositions and methods that include use of human pheromones to achieve various desirable non-behavioral responses, and especially an increase in exercise strength, exercise stamina, and/or a reduction in airway inflammation.

In one aspect of the inventive subject matter, an article includes a container with a human steroid pheromone in a formulation that allows the steroid pheromone to disperse from the container at a rate effective to elicit a response from a human exposed to the pheromone, wherein the response is selected from the group consisting of increased exercise strength, increased exercise stamina, and reduced airway inflammation. Such articles typically further include an information associated with the container that the formulation produces the response.

Thus, and viewed from another perspective, a method of providing over-the-counter relief of airway inflammation will include a step of providing the article, and advertising that the article reduces airway inflammation (e.g., sinusitis). Alternatively, or additionally, a method of providing an over-the-counter anabolic formulation may include a step of providing the article, and advertising that the article increases at least one of exercise strength and exercise stamina.

It is generally preferred that the pheromone has an androst-16-en structure, and in most preferred aspects, the pheromone is 5α-androst-16-en-3-one (androstenone), 5α-androst-16-en-3α-ol (androstenol), and/or 5α-androst-16-en-3β-ol (epi-androstenone). Typically, the pheromone is present in a composition in a predominant proportion (e.g., at least 60-90 wt %), is in a dry formulation, and may further include an odorant. While various containers are deemed suitable for gradual release of the pheromone, especially preferred containers include at least one wall that is fabricated from (high-density) polyethylene or polypropylene.

In further contemplated aspects of the inventive subject matter, the human pheromone is associated with an information that informs a user that exposure to the human pheromone will result in the desired non-behavioral effect. Most typically, such information is provided as a printed and/or displayed information, which may be on the container, a package in which at least part of the container is disposed, and/or on a sales brochure or flyer that is displayed in proximity (e.g., same sales stand, shelf, or store) to the product.

Various objects, features, aspects and advantages of the present invention will become more apparent from the following detailed description of preferred embodiments of the invention.

### DETAILED DESCRIPTION

The inventors have discovered that steroid pheromones, and especially human steroid pheromones can effectively achieve a desirable non-behavioral result where administered via inhalation. Among other contemplated desired results, especially preferred results include an increase in exercise strength, exercise stamina, and a reduction or even elimination of airway inflammation (and especially sinusitis).

The term “steroid pheromone” as used herein refers to a natural or synthetic compound having a steroid scaffold (cyclopentanoperhydrophenanthrene ring system) with a plurality of substituents and optional double bonds, wherein the compound elicits in vivo a measurable response in the vomeronasal organ (e.g., measured as negative receptor binding potential as described in U.S. Pat. No. 5,272,134, incorporated by reference herein) of an animal having a vomeronasal organ. The term “human steroid pheromone” as also used herein refers to a steroid pheromone that elicits an in vivo measurable response in the human vomeronasal organ.

Thus, especially preferred human steroid pheromones include various 16-androstenes and estrenes. It should be noted, that the terms “steroid pheromone” and “human steroid pheromones” expressly exclude corticosteroids (i.e., those produced in the cortex of the adrenal gland), and steroids with a testosterone scaffold (i.e., 17-Hydroxy-(17-beta)-androst-4-en-3-one). As still further used herein the term “about” in conjunction with a numeral refers to a range of +/−10% of that numeral, inclusive.

In one exemplary aspect of the inventive subject matter, the inventor placed about 500 mg of 5α-androst-16-en-3-one (androstenone) in a polyethylene plastic bag and placed the bag on top of a desk away from direct sunlight in an air-conditioned room of about 76° F. Remarkably, after about five to seven days of exposure (lasting in total between two to eight hours per day) to the human pheromone, all signs and symptoms of a previously diagnosed sinusitis had disappeared. Moreover, at about the same time, the inventor recognized that based on his previous exercise regimen, his exercise strength and stamina had substantially increased. For example, increased strength was evidenced by an at least 5-10%, and more typically 10-20% increase of weight that was lifted as compared to the same exercise before exposure to the pheromone. Similarly, the number of repeats per exercise were significantly increased, most typically by between about 5-20%.

With respect to the particular human steroid pheromone, it should be noted that numerous steroids other than androstenone (see Formula 1A) may also be suitable, and it is generally contemplated that all other human steroid pheromones are also considered appropriate. However, particularly preferred alternative human steroid pheromones include those having an androst-16-en structure, and especially those as depicted in Formulae 1B and 1C below.

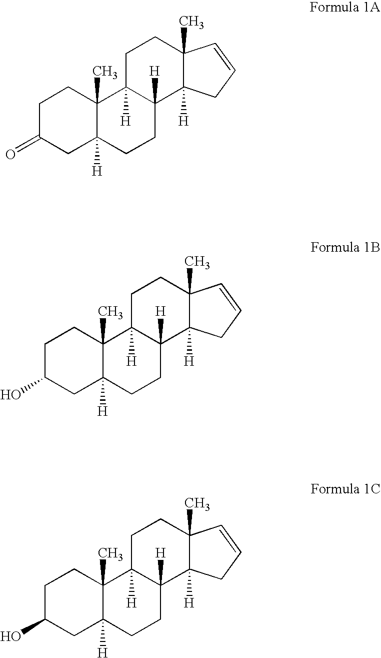

Still further suitable steroid pheromones also include those not necessarily characterized as human steroid pheromones, and exemplary additional steroid pheromones include 16-steroid compounds according to Formula 2 below 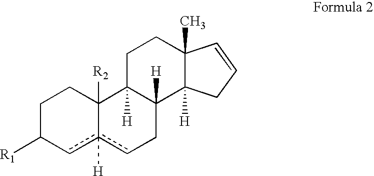
in which R₁ is oxo, alpha-hydroxy, and beta-hydroxy; R₂ is halogen, hydrogen, hydroxy, acyl, acyloxy, alkoxy, lower alkyl, methyl, hydroxyalkyl, hydroxymethyl, acyloxyalkyl, alkoxyalkyl, acyloxymethyl, and alkoxymethyl; [symbol] is an optional single bond; and [symbol] is an optional double bond.

It should further be appreciated that such compounds may have their substituents R₁ and R₂ independently in alpha or beta orientation, and that the double bonds in the ring system may be present in the alternative. Depending on the double bond, the optional hydrogen may or may not be present. Thus, and among other compounds, suitable 16-androstenes will include 4,16-androstadien-3-one, 19-hydroxy-4,16-androstadien-3-one, 4,16-androstadien-3α (or β)-ol, 19-nor-4,16-androstadien-3-one, 19-nor-10-OH-4,16-androstadien-3-one, 19-hydroxy-4,16-androstadien-3-one, 5,16-androstadien-3β-ol, 5α-5,16-androstadien-3α-ol, 19-nor-16-androsten-3-one, 19-nor-16-androsten-3α-ol, and 19-nor-16-Androsten-3β-ol.

In less preferred aspects of the inventive subject matter, estrene steroid pheromones are also contemplated, and exemplary suitable estrene steroid pheromones are described in U.S. Pat. No. 5,278,141, which is incorporated by reference herein. Additionally, it is contemplated that the pheromones according to the inventive subject matter may also exert their desired activity as metabolites, and/or as oxidized or otherwise transformed compounds (which may be generated by oxidation or other process during exposure to the environment). Still further, it should be recognized that more than one steroid pheromone may be used in conjunctions with the teachings presented herein, and that all reasonable combinations of two or more steroid pheromones are deemed suitable.

Suitable amounts of contemplated steroid pheromones may vary considerably, and it is generally contemplated that all amounts are appropriate so long as such amounts will provide at least some effect to a person. Therefore, in alternative aspects of the inventive subject matter, the pheromones contemplated herein may be present in an amount of between about 1 mg and 1000 mg, and even more. However, and depending on the intended duration of use, it is generally preferred that the amount of steroid pheromones will typically be in the range of between about 10 mg and 500 mg. Of course, it should be recognized that the steroid pheromone may be present as a relatively pure compound, or in a mixture of two or more components. Among other suitable ingredients, fragrance, carriers, and solvents are especially contemplated.

In yet further contemplated aspects, the steroid pheromones according to the inventive subject matter may be provided as synthesized (typically at a purity of at least 90%, and more typically of at least 95%), and thus be in a dry crystalline or powder formulation. However, in alternative aspects, the pheromones may also be dissolved or dispersed in one or more solvents, and most preferably hydrophobic solvents. Where a solvent is employed, it is generally preferred that the boiling point of the solvent is at a temperature well above room temperature (e.g., at least 35° C., more typically at least 50° C.), however, low-boiling point solvents or mixtures are not expressly excluded. Depending on the particular formulation, it is also contemplated that a carrier may be employed to improve one or more release characteristics (e.g., pheromone in a liquid formulation on a woven carrier strip, or dry formulation on aerosolizable powder). Typically, however, the pheromone is present in the (preferably dry) composition in an amount of at least 60 wt %, more typically at least 80 wt %, and most typically at least 90 wt %.

Therefore, it should be particularly recognized that the rate of release (i.e., amount of pheromone available from a container in a standard cubic meter of air over 24 hours) may be controlled using numerous factors, including surface area of the pheromone preparation, type of solvent (if present), ambient temperature, air agitation, type of pheromone, etc. However, it is generally preferred that the rate of release is typically in the range of human rate of release (i.e., amount of pheromone available from a human in a standard cubic meter of air over 24 hours) to about 10,000-fold of human release, more typically between 10-fold to 100-fold of the human release, and most typically between 50-fold to 1000-fold of the human release.

Furthermore, it is contemplated that the release dynamic can be static, or non-linear as desired. For example, in some cases, it may be desired that the release rate is held constant. On the other hand, increasing release rates may be implemented where a person desires to start of with a relatively low dosage, and decreasing release rates may be advantageous where a user may want to gradually discontinue exposure. Also contemplated non-linear release rates include circadian changes in release or seasonal changes.

Thus, it should be recognized that the container may have numerous configurations and features to accommodate various desired parameters. For example, in a conceptually relatively simple approach, the container may be a open or closed plastic bag (e.g., high-density or low density polyethylene or polypropylene) of substantially the same volume as the pheromone, wherein the bag may have additional openings, pores, or portions otherwise permeable for the steroid pheromone. On the other hand, suitable containers may also be more complex and include dedicated areas of storage for the pheromones (e.g., multiple shelves to increase overall surface exposure to the environment). Alternatively, containers may also be configured to receive a carrier or carrier compositions that expose the pheromone to the environment. Where desirable, the container may further include additional one or more implements to control the release rate of the pheromone. For example, and as described above, contemplated containers may include a thermal control element that is configured to raise, lower and/or maintain a particular temperature of the steroid pheromone. Alternatively, and especially where the pheromone is in a liquid formulation, a vent may be included to assist evaporation of the solvent and the pheromone into the atmosphere.

Therefore, it should be appreciated that the compounds according to the inventive subject matter will typically be transferred (e.g., as isolated molecules, droplets, emulsions, or aerosol) into the surrounding atmosphere without the use of a propellant (e.g., as those known in spray cans, including optionally halogenated hydrocarbons). Viewed from another perspective, it is generally preferred that the release rate from the container is predominantly (i.e., at least 70%) determined by the rate of evaporation (of the solvent and/or the steroid pheromone). Therefore, and in contrast to directly inhaled products (e.g., from an aerosol container), the duration of administration of contemplated pheromones is most commonly identical with the duration of exposure and/or proximity to the container. Preferably, the container is therefore configured to allow continuous administration of the pheromone over a period of at least 60 minutes, more typically at least 2 hours, and most typically at least 4 hours (and even longer). Overall suitable periods of administrations are typically between 1 day and several weeks, which may be continuous with the same pheromone, or discontinuous or in a cycling pattern using one, two, or more steroid pheromones.

With respect to the effects afforded by contemplated compositions and devices, it is generally contemplated that the steroid pheromones will exert both a desirable behavioral effect as well as a desirable non-behavioral effect. Among other things, the inventor has discovered that upon exposure to the contemplated compounds and composition, his exercise strength has significantly improved. The term “exercise strength” as used herein refers to an ability to lift a particular weight in a given exercise routine (e.g., lifting of 100 pounds on a bench press). Thus, where exercise strength is increased, heavier weights can be lifted. Most typically the increase in exercise strength is between 5 and 40%, and more typically between 10 and 25%. Similarly, the inventor has also discovered that upon exposure to the contemplated compounds and composition, his exercise stamina has significantly improved. The term “exercise stamina” as used herein refers to an ability to lift a particular weight in a given exercise routine for a maximum of repeats Rₘₐₓ (e.g., lifting of 100 pounds on a bench press ten times). Thus, where exercise stamina is increased, the weight can be lifted more that Rₘₐₓ times. Most typically the increase in exercise strength is between 5 and 30%, and more typically between 10 and 20%.

Still further, the inventor has noted that a previously diagnosed and persistent sinus inflammation significantly improved, and eventually disappeared. Therefore, it is also contemplated that the compositions and methods presented herein may present a convenient matter to treat and/or alleviate signs and symptoms of airway inflammation. Most preferably, such inflammation may be sinusitis, asthma, and allergic airway inflammation. In yet other contemplated aspects, additional behavioral effects may be advantageously conferred by use of contemplated compositions and methods, especially including feeling of increased energy, sexual desire, and self-confidence.

Thus, specific embodiments and applications of pheromone compositions and methods have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. An article of manufacture comprising:
a container that comprises a human steroid pheromone in a formulation that allows the steroid pheromone to disperse from the container at a rate effective to elicit a response from a human exposed to the pheromone;
wherein the response is reduced airway inflammation; and
an information associated with the container that the formulation produces the response.

2. The article of claim 1 wherein the pheromone has an androst-16-en structure.

3. The article of claim 1 wherein the pheromone is selected from the group consisting of 5α-androst-16-en-3-one, 5α-androst-16-en-3α-ol, and 5α-androst-16-en-3β-ol.

4. The article of claim 1 wherein the formulation comprises at least 90 wt % of the human steroid pheromone.

5. The article of claim 1 wherein the formulation is a dry formulation.

6. The article of claim 1 wherein the container comprises at least one wall manufactured from polyethylene or polypropylene.

7. The article of claim 1 wherein the airway inflammation is sinusitis.

8. The article of claim 1 wherein the information is a printed information on at least one of the container and a packaging for the container.

9. A method of providing over-the-counter relief of airway inflammation, comprising a step of providing the article of claim 1, and advertising that the article reduces airway inflammation.

10. The method of claim 9 wherein the pheromone has an androst-16-en structure.

11. The method of claim 9 wherein the pheromone is selected from the group consisting of 5α-androst-16-en-3-one, 5α-androst-16-en-3α-ol, and 5α-androst-16-en-3β-ol.

12. The method of claim 9 wherein the airway inflammation is sinusitis.

13. The method of claim 9 wherein the step of advertising comprises providing a printed information on at least one of the container and a packaging for the container.

14. The method of claim 9 wherein the formulation in the article comprises at least 90 wt % of the human steroid pheromone.

15. (canceled)

16. (canceled)

17. (canceled)

18. (canceled)

19. (canceled)

20. (canceled)
